(19) United States
(12) Patent Application Publication
(10) Pub. No.: US 2022/0062477 A1
(43) Pub. Date: Mar. 3, 2022

(54) CART SANITIZING APPARATUS AND METHOD

(71) Applicant: Banks Machine and Engineering, Inc., Shelbyville, IN (US)

(72) Inventors: Adam Hunt, Greenfield, IN (US); Kent Colclazier, Greenfield, IN (US)

(21) Appl. No.: 17/461,089

(22) Filed: Aug. 30, 2021

Related U.S. Application Data
(60) Provisional application No. 63/071,678, filed on Aug. 28, 2020.

(51) Int. Cl. A61L 2/10; B08B 13/00; B08B 5/02; A61L 2/22; B25J 11/00; G05D 1/02; B65F 7/00

(52) U.S. Cl. CPC A61L 2/10; B08B 13/00; B08B 5/023; A61L 2/22; B25J 11/0085; G05D 1/0225; H04N 7/18; A61L 2202/11; A61L 2202/122; A61L 2202/17; B65F 2210/168; B65F 2210/1443; B65F 7/00

(57) ABSTRACT

A cart sanitizing system that has a conveyor assembly configured to selectively move a cart and a cleaning chamber assembly configured to selectively clean a cart positioned therein. The conveyor assembly repositions a cart into the cleaning chamber for a cleaning process and removes the cart from the cleaning chamber after the cleaning process is complete.

### CROSS-REFERENCE TO RELATED APPLICATION

The present disclosure claims the benefit of U.S. Provisional Application No. 63/071,678 filed Aug. 28, 2020, the contents of which are incorporated herein by reference in entirety.

### FIELD OF THE DISCLOSURE

This disclosure relates generally to an automatic system for collecting, transporting, inspecting, cleaning, and sanitizing a used cart and presenting the clean cart to the consumer for use.

### BACKGROUND

Mobile shopping carts are typically shared by many different and unrelated users. Each user typically manually pushes or pulls the shopping cart throughout a corresponding store, thereby depositing any germs or the like of the user on the shopping cart. As such, there is potential for a person to transfer germs to a subsequent user through contact with the shopping cart.

Guided cart collection assist vehicles are used by retailers to reduce employee fatigue caused by manual handling of large quantities of carts. Cart collection assist vehicles provide a motorized unit that pushes one or more stacked carts from the back while a user guides the front of the cart stack. The user communicates with the cart collection assist vehicle via a handheld remote or the like to identify when a pushing force is desired. The typical guided cart collection assist vehicle is intended to reduce time and labor for employees to return used carts to the store for reuse.

Further, techniques for manually cleaning single carts or semi-automatically cleaning single carts are available. Such techniques involve a user positioning a cart in a sanitizing bay and engaging a sanitizing process. However, there is a need for an automatic system to keep communal items clean, disinfected, and safe for the consumer.

### SUMMARY

One embodiment of this disclosure is a cart sanitizing system that has a conveyor assembly configured to selectively move a cart and a cleaning chamber assembly configured to selectively clean a cart positioned therein. The conveyor assembly repositions a cart into the cleaning chamber for a cleaning process and removes the cart from the cleaning chamber after the cleaning process is complete.

One example of this embodiment has an unmanned ground vehicle configured to selectively position one or more cart along the conveyor assembly. Another example uses a camera system for detecting debris in a cart on the conveyor assembly. Part of this example may include a robotic arm configured to selectively remove debris identified by the camera system.

Another example of this embodiment has a fan assembly directing air up from a bottom area to remove debris from a cart on the conveyor assembly. Part of this example has a trash collection reservoir wherein the fan assembly directs air flow and any corresponding debris to the trash collection reservoir. In one aspect of this part, the trash collection reservoir comprises a sensor that indicates when the trash collection reservoir is full.

Yet another example of this disclosure includes a status indicator couplable to a cart. The status indicator provides an indication the cart is clean when the cart has passed through the cleaning chamber assembly. In part of this example, the status indicator may also provide an indication that the cart is dirty when the cart exits an adjacent store.

In another example, the cleaning chamber assembly uses UV-C disinfecting lighting arrays to disinfect a cart positioned therein. In yet another example, the cleaning chamber uses a sprayed or atomized disinfecting solution for the cleaning process.

Another embodiment of this disclosure is a method for cleaning a cart that includes aligning a cart with a conveyor assembly, selectively moving the cart with the conveyor assembly into a cleaning chamber assembly, executing a cleaning process in the cleaning chamber with a cart positioned therein, and removing the cart from the cleaning chamber with the conveyor assembly after the cleaning process is complete.

One example of this embodiment includes controlling an unmanned ground vehicle to selectively position one or more cart along the conveyor assembly. Another example includes detecting debris in the cart with a camera system while the cart is on the conveyor assembly. Part of this example may include selectively removing debris from the cart identified by the camera system with a robotic arm.

Yet another example of this embodiment includes directing air through the cart with a fan assembly to remove debris from the cart while on the conveyor assembly and directing the debris to a trash collection reservoir. Another example includes altering a reading on a status indicator coupled to the cart when the cart has passed through the cleaning chamber assembly. Part of this example may include altering the reading on the status indicator coupled to the cart when the cart has been used by a user.

Another example of this embodiment includes using UV-C disinfecting lighting arrays for the cleaning process. Yet another example includes using a sprayed or atomized disinfecting solution for the cleaning process.

### DRAWINGS

The above-mentioned aspects of the present disclosure and the manner of obtaining them will become more apparent and the disclosure itself will be better understood by reference to the following drawings:

FIG. 1 is a elevated perspective view of a cart sanitizing system;

FIG. 2 is a side view of the cart sanitizing system of FIG. 1;

FIG. 3 is a top view of another embodiment of the cart sanitizing system;

FIG. 4 is a partial back-side view of a cart having a status indicator; and

FIG. 5 is a schematic block diagram illustrating some of the components of the present disclosure.

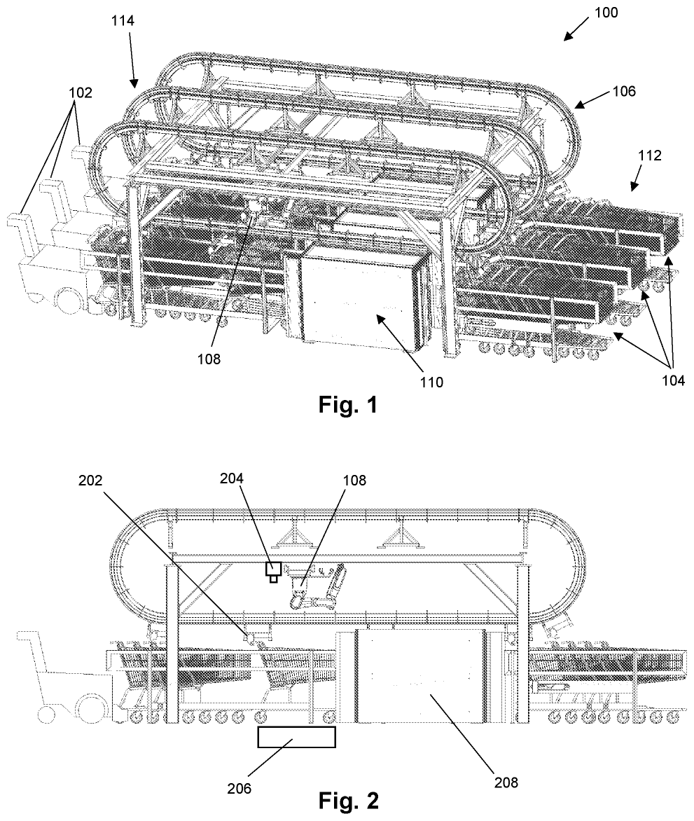

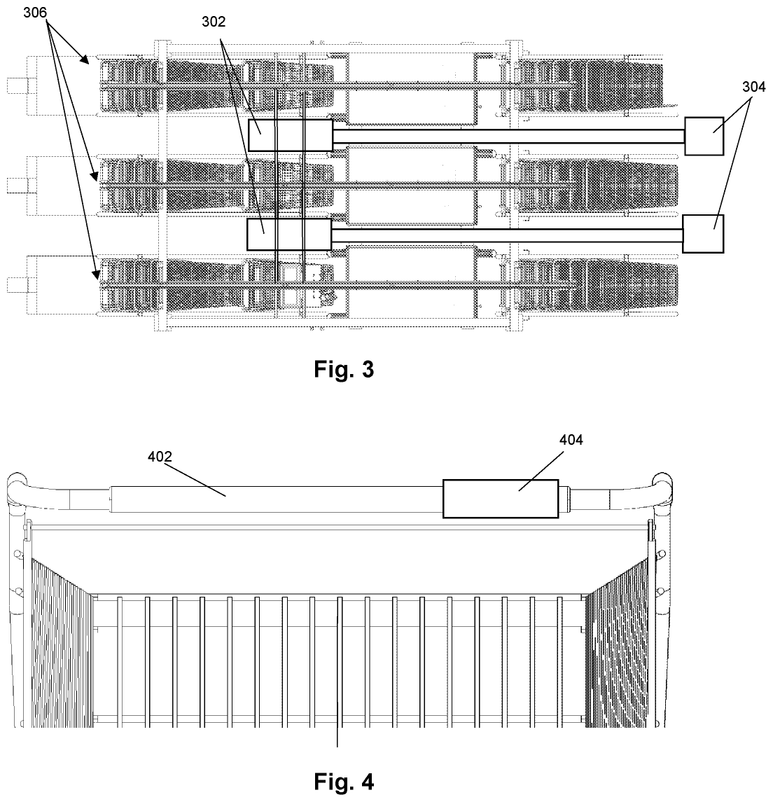

Corresponding reference numerals are used to indicate corresponding parts throughout the several views.

### DETAILED DESCRIPTION

The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure.

One aspect of this disclosure includes an automatic system that will be sized to be installed in the standard size cart corrals attached near the entrance of a corresponding store. At least one embodiment is a cart sanitizing system 100, the system 100 will include an employee assisted cart moving device such as an Unmanned Ground Vehicle (“UGV 102”) 102. These UGVs 102 may communicate with the cart sanitizing system 100 to push one or more carts 104 to a desired location. More specifically, the cart sanitizing system 100 may communicate wirelessly with the UGV 102 to take motion control of the UGV 102 to safely drive and guide a single cart or group of stacked carts into a coral to be coupled to a coupler 202 of a conveyor assembly 106. Further, the cart sanitizing system 100 may be capable of notifying the operator that a UGV 102 has completed the task of pushing a line of carts 104 safely into the conveyor assembly 106. The notification may be sent to a user wirelessly through known wireless communication protocols or shown on a display on the cart sanitizing system 100. Regardless, the notification indicates that the UGV 102 is available to make another collection run to the parking lot among other things.

The cart sanitizing system 100 may include a complete sanitizing system that may be able to automatically inspect the carts 104 for items, trash, or visual surface contamination via a camera system 204 directed towards the carts as they are moved along by the conveyor assembly 106. The image data provided by the camera system 204 may be processed to provide vision assisted guidance for one or more multi-axis robotic arm 108. The robotic arm 108 may be slidable along rails within the conveyor assembly 106 to provide axis to any portion of carts 104 passing there through. The cart sanitizing system 100 may utilize custom algorithms and/or artificial intelligence routines to process the camera system 204 data and manipulate the robotic arm 108 to clean debris and the like from carts 104 passing thereunder.

The cart sanitizing system 100 may also utilize the robotic arm 108 to wipe a push-bar 402 of the cart as part of the cleaning process. The robotic arm 108 may have a cleaning agent on a cleaning pad that is then wiped along the push bar 402 of the cart to substantially disinfect the common contact surface of the push bar 402.

Further, the cart sanitizing system 100 may automatically remove trash, debris, or soiled supplies located in the cart 104 as it moves along the conveyor assembly 106. In one non-exclusive example, a high-powered stream of air may be directed from a fan assembly 206 underneath the cart towards a collection conduit 302 adjacent to the cart 104. The stream of air produced by the fan assembly 206 may carry debris out of the cart 104 and into the collection conduit 302 where it may be directed to a trash collection reservoir 304 or other collection location. In one example, the fan assembly 206 may draw air through a filter in the trash collection reservoir 304 to blow up through the cart 104. The trash collection reservoir 304 may further be fluidly coupled to the collection conduit 302 wherein the collection conduit 302 provides a vacuum of air into the trash collection reservoir 304 to thereby direct trash and other debris out of the cart 104 via the fan assembly 206 and route the trash or debris into the trash collection reservoir 304 because of the vacuum created by the fan assembly 206 intake. In one non-exclusive example, the trash collection reservoir 304 may have one or more sensor identifying the remaining capacity of the trash collection reservoir 304 to thereby provide an automatic notification when the trash collection reservoir 304 is full.

The cart sanitizing system 100 may utilize the conveyor assembly 106 to direct the cart 104 through a germ-killing area or cleaning chamber 110 to disinfect the cart 104. The cleaning chamber 110 may include UV-C disinfecting lighting arrays, sprayed or atomized solvents, mechanical cleaners, or any other known germ-killing protocol. In one non-exclusive example, the cleaning chamber 110 will be contained within a safety enclosure 208 to eliminate any safety concerns to employees or end-users in the area. If the cart 104 is cleaned with a liquid agent, the cart will also be dried by one or more of a timed, blown, and/or heat-dried process before being presented for use. Once the cart 104 is through the cleaning chamber, the cart 104 may be presented to the store customers for use at an exit 112 of the cart sanitizing system 100.

In one aspect of this disclosure, the cart sanitizing system 100 may define one or more entry lane 306 along an entrance side 114 of the cart sanitizing system 100. The entry lanes 306 are sized to receive stacks of carts 104 and guide the carts 104 towards the cleaning chamber 110. As one non-exclusive example, the UGV 102 may be controlled by the cart sanitizing system 100 to direct the stack of carts 104 into the entry lane 306. Once within the entry lane 306, the coupler 202 on the conveyer assembly 106 may contact the push bar 402 or other location of the cart 104 to control movement of the cart 104 into the cleaning chamber 110 and ultimately to the exit 112. The cart sanitizing system 100 may communicate with both the UGV 102 and the conveyor assembly 106 to control the positioning of the cart within the corresponding lane 306. In other words, as large stack of carts 104 may have a UGV 102 at one end pushing the carts 104 towards the conveyor assembly 106 and stop pushing the carts 104 once a cart 104 is engaged by the coupler 202 of the conveyor assembly 106. When the conveyor assembly 106 is ready for another cart 104, the UGV 102 may advance the stack of carts in the entry lane 306 sufficiently so the coupler 202 of the conveyor assembly 106 may engage the next cart 104 in line.

The coupler 202 of the conveyor assembly 106 may separate a leading cart from a stack of carts in the entry lane 306. The separated cart may pass through the high-powered stream of air from the fan assembly 206 and any debris therein may be blown out of the cart 104 and captured by the collection conduit 302 and directed to the dedicated trash collection reservoir 304. The robotic arm 108 may also wipe the push bar 402 and any other portion of the cart 104 while the cart 104 is separated from the stack of carts in the entry lane 306 and waiting to enter the cleaning chamber 110.

The conveyor assembly 106 may direct the separated cart 104 into the cleaning chamber 110. At this point, loose debris has been removed and the push bar 402 has been cleaned. However, the cleaning chamber 110 may substantially disinfect the remaining portions of the cart 104 when the cart 104 is positioned therein. The conveyor assembly 106 may direct the separated cart 104 into the cleaning chamber 110 and hold the cart at that location for a cleaning cycle executed by the cleaning chamber 110. In one aspect of this disclosure, the cleaning chamber 110 utilizes Ultraviolet C type lights to disinfect the cart 104 once positioned therein. In this embodiment, the cleaning chamber 110 may have entrance and exit doors that are automatically opened and closed to position the cart 104 therein. More specifically, the doors may substantially prevent ultraviolet light from shining outside of the cleaning chamber 110.

While a particular type of cleaning chamber 110 is discussed herein, any cleaning chamber 110 capable of disinfecting a cart is considered, and the ultraviolet cleaning chamber discussed herein is just one example. Other cleaning chambers considered herein may utilize a sprayed and/or atomized cleaning agent among other things. Accordingly, many cleaning methods are considered herein for the cleaning chamber 110.

After the cleaning chamber 110, the conveyer assembly 106 may direct the cart to an exit lane on the exit side 112 of the cart sanitizing system 100. In the exit lane, the sanitized carts 104 may be repositioned in stacks for users to grab. Once repositioned in the stack, the coupler 202 of the conveyer assembly 106 may release the cart 104 so a user may grab the disinfected cart 104 for use.

In one aspect of this disclosure, while the cart 104 is in or exiting the cleaning chamber 110, a status indicator 404 on the cart 104 may be altered to represent the cart 104 has been cleaned. The status indicator 404 may be a digital display, a manual slide or flap, a color indicator, or any other indicator capable of communicating to a user that the cart 104 has been cleaned. The status indicator 404 may also be part of a counterfeit sensor assembly for the corresponding store. In this embodiment, once the user enters the store through a sensing portal, the status indicator 404 may identify the movement of the cart and transition to indicate the cart 104 is now in use. Alternatively, the status indicator 404 may switch to indicate the cart 104 is dirty if the cart leaves a proximity of the cart sanitizing system 100 or when the cart 104 leaves the exit 112 lane. Alternatively, the status indicator 404 may indicate the cart 104 is dirty after a preset time has expired after cleaning. Accordingly, the status indicator 404 may indicate when the cart 104 has been through a cleaning process in the cleaning chamber 110 and when the cart 104 may be dirty and require cleaning.

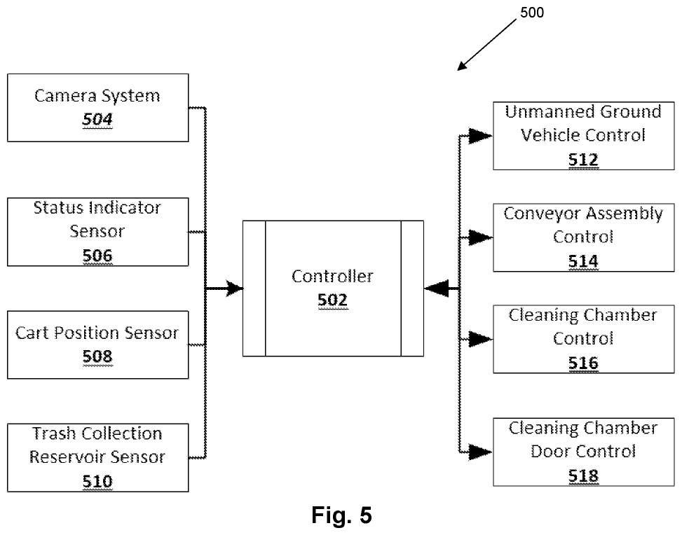

Referring now to FIG. 5, a schematic representation 500 of some of the components of the cart sanitizing system 100 are illustrated. More specifically, a controller 502 may receive signals from sensors and send commands or otherwise control components of the cart sanitizing system 100. The controller 502 may have a processor capable of processing data and a memory unit capable of storing and manipulating data and the like. The controller 502 may be located on the cart sanitizing system 100 or remotely. Further, the controller 502 may comprise many controllers each communicating with the other to execute the functionality discussed herein.

The controller 502 may receive and manage signals from a camera system 504. The camera system 504 may be the camera system 204 and may include additional camera systems as well. Regardless, the camera system 504 may provide visual input to the controller 502 to be processed be the controller 502 to determine whether there is debris in one or more of the carts 104, the positioning of the carts 104, whether there is an obstruction in the cleaning chamber 110, or any other information obtainable from a camera system.

The controller 502 may also communicate with and control a status indicator sensor 506 to determine and alter the status displayed on the status indicator 404. In this example, when the cart 104 enters the cleaning chamber 110 the status indicator sensor 506 may identify that the cart is being cleaned via the controller 502. Further, the controller 502 may then alter the status displayed on the status indicator 404 to show the cart 104 was cleaned once the cleaning process is complete. Further still, the status indicator sensor 506 may be a proximity sensor wherein when the cart 104 is outside of the proximity, the status indicator 404 illustrates that the cart 104 is dirty.

The controller 502 may also communicate with one or more cart position sensor 508 to identify the location of the carts 104 in the corresponding lanes. The controller 502 may then use the cart position information to manipulate the conveyor assembly 106 so the coupler 202 can properly engage a cart 104. Further, the controller 502 may communicate with a trash collection reservoir sensor 510 to identify when the trash collection reservoir 304 is full. The trash collection reservoir sensor 510 may identify the weight of the trash collection reservoir 304 or the height of debris therein so the controller 502 can determine when the trash collection reservoir 304 is full.

The controller 502 may also have unmanned ground vehicle control 512 that allows the controller 502 to communicate with, and control, one or more UGV 102. The controller 502 may communicate with UGV 102 to move a line of carts into corresponding entrance lanes 114 to be engaged by the coupler 202 of the conveyor assembly 106. The controller 502 may utilize one or more of the camera system 504 and cart position sensor 508 to determine how far to move the carts 104 with the unmanned ground vehicle controller 512.

The controller 502 may also communicate with conveyor assembly control 514. The controller 502 may monitor the position of the carts 104 with the camera system 504 or the cart position sensor 508 to selectively advance carts into the cleaning chamber 110. The controller 502 may utilize the conveyor control assembly 514 to selectively position carts in the cleaning chamber 110, allow the cleaning process to complete, then move the cart 104 to the exit 112.

The controller 502 may also have cleaning chamber control 516. The cleaning chamber control 516 may allow the controller 502 to selectively initiate a cleaning process of the cleaning chamber 110. The cleaning process may be engaging Ultraviolet C lights to disinfect any cart 104 in the cleaning chamber 110 or spray or atomize a cleaning agent on any cart 104 located in the cleaning chamber 110. As part of the cleaning chamber control 516, the controller 502 may also have cleaning chamber door control 518. The cleaning chamber door control 518 may allow the controller 502 to selectively open and close doors on the cleaning chamber 518 to protect the areas around the exterior of the cleaning chamber 110 from ultraviolet light exposure or overspray of cleaning agents among other things. The cleaning chamber door control 518 may open the corresponding cleaning chamber doors to allow a cart 104 to enter and leave the cleaning chamber 110 and close the doors when the cart 104 is therein and being cleaned.

Further, sensors may be positioned around the cleaning chamber 110 to determine when an unknown object is detected. In this configuration, the cleaning chamber 110 may not initiate a cleaning process when an unknown object is detected. This safety feature may prevent harm to the automatic cleaning system or users among other things.

The cart sanitizing system 100 illustrated herein has three separate lanes. However, this disclosure contemplates cart sanitizing systems 100 with fewer or more lanes as well. A person skilled in the art understands the teachings of this disclosure can be applied to any number of lanes for cleaning a cart.

While a particular form of the invention has been illustrated and described, it will be apparent that various modifications can be made without departing from the spirit and scope of the invention. For example, the system may be adapted to be used for hand held baskets, small double basket carts, special carts for kids, and motorized carts for the handicap among other things. Accordingly, it is not intended that the invention be limited, except as by the appended claims.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. In addition, the teachings of the disclosure provided herein can be applied to other systems, not necessarily the system described above. The elements and acts of the various embodiments described above can be combined to provide further embodiments.

While embodiments incorporating the principles of the present disclosure have been described hereinabove, the present disclosure is not limited to the described embodiments. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A cart sanitizing system, comprising:
a conveyor assembly configured to selectively move a cart; and
a cleaning chamber assembly configured to selectively clean a cart positioned therein;
wherein, the conveyor assembly repositions a cart into the cleaning chamber for a cleaning process and removes the cart from the cleaning chamber after the cleaning process is complete.

2. The cart sanitizing system of claim 1, further comprising an unmanned ground vehicle configured to selectively position one or more cart along the conveyor assembly.

3. The cart sanitizing system of claim 1, further comprising a camera system for detecting debris in a cart on the conveyor assembly.

4. The cart sanitizing system of claim 3, further comprising a robotic arm configured to selectively remove debris identified by the camera system.

5. The cart sanitizing system of claim 1, further comprising a fan assembly directing air up from a bottom area to remove debris from a cart on the conveyor assembly.

6. The cart sanitizing system of claim 5, further comprising a trash collection reservoir wherein the fan assembly directs air flow and any corresponding debris to the trash collection reservoir.

7. The cart sanitizing system of claim 6, wherein the trash collection reservoir comprises a sensor that indicates when the trash collection reservoir is full.

8. The cart sanitizing system of claim 1, further comprising a status indicator couplable to a cart, wherein the status indicator provides an indication the cart is clean when the cart has passed through the cleaning chamber assembly.

9. The cart sanitizing system of claim 8, wherein the status indicator provides an indication that the cart is dirty when the cart exits an adjacent store.

10. The cart sanitizing system of claim 1, further wherein the cleaning chamber assembly uses UV-C disinfecting lighting arrays to disinfect a cart positioned therein.

11. The cart sanitizing system of claim 1, further wherein the cleaning chamber uses a sprayed or atomized disinfecting solution for the cleaning process.

12. A method for cleaning a cart, comprising:
aligning a cart with a conveyor assembly;
selectively moving the cart with the conveyor assembly into a cleaning chamber assembly;
executing a cleaning process in the cleaning chamber with a cart positioned therein; and
removing the cart from the cleaning chamber with the conveyor assembly after the cleaning process is complete.

13. The method for cleaning a cart of claim 12, further comprising controlling an unmanned ground vehicle to selectively position one or more cart along the conveyor assembly.

14. The method for cleaning a cart of claim 12, further comprising detecting debris in the cart with a camera system while the cart is on the conveyor assembly.

15. The method for cleaning a cart of claim 14, further comprising selectively removing debris from the cart identified by the camera system with a robotic arm.

16. The method for cleaning a cart of claim 12, further comprising directing air through the cart with a fan assembly to remove debris from the cart while on the conveyor assembly and directing the debris to a trash collection reservoir.

17. The method for cleaning a cart of claim 12, further comprising altering a reading on a status indicator coupled to the cart when the cart has passed through the cleaning chamber assembly.

18. The method for cleaning a cart of claim 17, further comprising altering the reading on the status indicator coupled to the cart when the cart has been used by a user.

19. The method for cleaning a cart of claim 12, further comprising using UV-C disinfecting lighting arrays for the cleaning process.

20. The method for cleaning a cart of claim 12, further comprising using a sprayed or atomized disinfecting solution for the cleaning process.
